(19) United States
(12) Patent Application Publication
(10) Pub. No.: US 2014/0335077 A1
(43) Pub. Date: Nov. 13, 2014

(54) Compositions and Methods for the Treatment of Cancer Using IGF-IR Antagonists and MAPK/ERK Inhibitors

(71) Applicants: Leonard Girnita, Taby (SE); Ada Girnita, Taby (SE)

(72) Inventors: Leonard Girnita, Taby (SE); Ada Girnita, Taby (SE)

(21) Appl. No.: 14/270,789

(22) Filed: May 6, 2014

Related U.S. Application Data
(60) Provisional application No. 61/820,453, filed on May 7, 2013.

(51) Int. Cl. C07K 16/28; A61K 31/4709; A61K 39/395

(52) U.S. Cl. CPC C07K 16/2863; A61K 39/3955; A61K 31/4709
USPC 424/133.1; 514/312

(57) ABSTRACT

The present invention relates generally to the field of cancer therapy. More specifically the present invention relates a combination therapy where IGF-1R antagonists are combined with MAPK/ERK pathway inhibitors. The present invention relates to a therapeutic combination comprising an IGF-1R antagonist and a MAPK/ERK pathway inhibitor, and to methods for the production of an anti-cancer effect in a patient. The present invention relates to: a therapeutic combination comprising an IGF-1R antagonist and a MAPK/ERK pathway inhibitor; a combination product comprising an IGF-1R antagonist and a MAPK/ERK pathway inhibitor, a kit of parts comprising an IGF-1R antagonist and a MAPK/ERK pathway inhibitor; use of a therapeutic combination, combination product or kit of parts in the treatment of cancer; a method of treating cancer comprising administering the therapeutic combination, combination product or kit of parts to a patient.

### STATEMENT OF PRIORITY

This application claims the benefit, under 35 U.S.C. §119(e), of U.S. Provisional Application No. 61/820,453; filed May 7, 2013, the entire contents of which is incorporated by reference herein.

### FIELD OF THE INVENTION

The present invention relates generally to the field of cancer therapy. More specifically the present invention relates a combination therapy where IGF-1R antagonists are combined with MAPK/ERK pathway inhibitors.

### BACKGROUND OF THE INVENTION

Most forms of cancer arise through an evolutionary process that select the growth of clones and subclones of cells, which are less and less responsive to the normal intra- and extra-cellular growth control mechanisms. The multi-step or continuous development is known to involve changes at the genomic level (oncogene activation, loss of function of tumor suppressor genes, translocations resulting in fusion genes that encode chimeric proteins with tumorigenic functions, etc.). However, in the selection leading to cancer in somatic tissues the cancer cells make use of the normal extracellular signaling for proliferation and/or antiapoptosis to create a growth advantage over normal cells. Most of these signals are mediated by growth factors. Accumulated evidence suggests that members of the receptor tyrosine kinase (RTKs) ligand families including insulin-like growth factor (IGF), fibroblast growth factor (FGF), platelet derived growth factor (PDGF) and epidermal growth factor (EGF) play important roles in the development and progression of cancer. These growth factors regulate important cellular activities like growth, proliferation, differentiation and apoptosis or senescence; they mediate biological responses by binding and activating membrane-spanning receptors, which in turn transmit signals inside the cell.

The Insulin-like Growth Factor 1 Receptor (IGF-1R) is a protein found on the surface of human cells. The IGF-1R pathway is essential for the initiation, progression and metastasis of many cancers. In contrast to other receptor tyrosine kinases involved in cancer, IGF-1R is not frequently mutated or amplified.

Several lines of evidence implicate IGF-1R and its “classical” ligands IGF-1 and IGF-2 or the more recent discovered the antimicrobial peptide LL-37 in malignant transformation. Increased expression of IGF-1, IGF-1R or both has been documented in many human malignancies including carcinomas of the lung, breast, thyroid, gastrointestinal tract and prostate, as well as glioblastoma, neuroblastoma, melanomas, rhabdomyosarcoma, and leukemias. Prospective epidemiological studies identified high plasma levels of IGF-1 as a potential risk factor for several malignancies. In addition, the IGFs are potent mitogens for a wide range of tumor cell types in vitro. Furthermore, several oncogenes have now been shown to affect IGF-1 and IGF-1R expression. IGF-1R is involved not only in the induction of cell transformation but also in the maintenance of the transformed phenotype. IGF-1R was also identified as a positive regulator of the invasive/metastatic phenotype and IGF-1 as a paracrine growth-promoting factor for liver metastases.

The IGF system of ligands, receptors and binding proteins is undoubtedly a major player in normal cellular growth and differentiation, as well as in aberrant growth seen in neoplastic disorders. Whereas the IGFs and the IGF-1R are not by themselves oncogenes, experimental and epidemiological evidence suggest that they may enhance proliferation of preneoplastic and neoplastic cells.

### SUMMARY OF THE INVENTION

IGF-1R antagonists developed today have been shown not to be effective in clinical settings.

The inventors herein have surprisingly found that combining IGF-1R antagonist treatment with MAPK/ERK inhibitors provide improved treatment of cancer at least in the early stages of treatment.

Accordingly, the present invention discloses methods and compositions for treatment of cancer, comprising a combination therapy where IGF-1R antagonists are combined with MAPK/ERK pathway inhibitors.

In one embodiment, the present invention provides a composition comprising at least one insulin growth factor 1 receptor (IGF-1R) antagonist and at least one mitogen activated protein kinase/extracellular signal-regulated kinase (MAPK/ERK) pathway inhibitor.

In some embodiments, the present invention further provides a pharmaceutical composition comprising an IGF-1R antagonist, a MAPK/ERK pathway inhibitor and a pharmaceutically acceptable carrier. The present invention also discloses the use of this pharmaceutical composition.

The present invention further provides a method of treating cancer in a subject in need thereof, the method comprising administering to said subject a therapeutically effective amount of at least one insulin growth factor 1 receptor (IGF-1R) antagonist and at least one mitogen activated protein kinase/extracellular signal-regulated kinase (MAPK/ERK) pathway inhibitor.

In still other embodiments, treatment of cancer in a subject in need thereof with a therapeutically effective amount of an IGF-1R antagonist and a MAPPK/ERK pathway inhibitor provides an enhanced response to anti-IGF-1R therapies for all types of cancer that are dependent on IGF-1R expression.

The present invention relates to a therapeutic combination and administration schemes comprising an IGF-1R antagonist and a MAPK/ERK pathway inhibitor, and to methods for the production of an anti-cancer effect in a patient, which is accordingly useful in the treatment of cancer in a patient. More specifically in various embodiments, the present invention relates to: a therapeutic combination comprising an IGF-1R antagonist and a MAPK/ERK pathway inhibitor; a combination product comprising an IGF-1R antagonist and a MAPK/ERK pathway inhibitor, a kit of parts comprising an IGF-1R antagonist and a MAPK/ERK pathway inhibitor; use of a therapeutic combination, combination product or kit of parts in the treatment of cancer; a method of treating cancer comprising administering the therapeutic combination, a treatment design, combination product or kit of parts to a patient.

### BRIEF DESCRIPTION OF THE DRAWINGS

FIG. 1A-FIG. 1C. Effects of Figitumumab (CP-751871 (CP)) treatment on cell viability in ES cell lines. (A) Cell lysates were prepared from the indicated cell lines and analyzed by WB for total IGF-1R expression and GAPDH as a loading control. Signals were quantified by densitometry, normalized to GAPDH and displayed as a percentage of the total IGF-1R level in the CADO cell line. Data correspond to the mean±SEM from three independent experiments. (B) IGF-1R activity was analyzed in the indicated cell lines by measuring IGF-1 induced receptor auto-phosphorylation and the subsequent activation of the IGF-1R downstream signaling pathways MAPK/ERK and PI3K/AKT. Cells serum starved for 12 h were stimulated or not with IGF-1 (50 ng/ml) for 10 min and protein lysates analyzed by WB for phosphorylated IGF-1R (P-IGF-1R), phosphorylated ERK (P-ERK), phosphorylated AKT (P-AKT), ERK, AKT, IGF-1R and GAPDH. (C) Indicated cells were treated without and with 100 ng/ml CP for 48 h in the absence (SFM) or presence of 10% fetal bovine serum (Serum) and cell viability assayed by PrestoBlue reagent. Number of viable cells following CP treatment is displayed as percentage of untreated control. Data correspond to the mean±SEM from three independent experiments. Statistical analysis compared with SKBR3: *P<0.05, **P<0.01, **P<0.001.

FIG. 2A-FIG. 2D. Mechanism of CP induced IGF-1R downregulation: β-arrestin1 recruitment and receptor ubiquitination. (A) Cells incubated in serum free medium for 12 h were treated with 0, 0.1 or 1 μg/ml CP for 24 h. Protein lysates were analyzed by WB for IGF-1R and GAPDH as a loading control. (B) Cells were incubated in serum free medium for 12 h and stimulated with 100 ng/ml CP or 50 ng/ml IGF-1 for indicated times. Protein lysates were analyzed by WB for IGF-1R and GAPDH as a loading control. Signals were quantified by densitometry, normalized to GAPDH and expressed as a percentage of the IGF-1R at 0 h. Data correspond to the mean±SEM from three independent experiments. (C) Cells were incubated in serum free medium for 12 h and were unstimulated or stimulated with either IGF-1 or CP for 10 min. IGF-1R was immunoprecipitated from lysates and analyzed by WB (IB) for ubiquitination (Ub) and IGF-1R as a loading control. Molecular weight markers in KDa are indicated to the left of the panels. (D) β-Arrestin1 (β-arr1) was immunoprecipitated from cell lysates prepared as for (C). Immunoprecipitated proteins were analyzed by WB for IGF-1R. The whole lysates were analyzed by WB for GAPDH as a loading control.

FIG. 3A-FIG. 3D. β-Arrestin1 dependence of CP induced IGF-1R degradation MEF and MEF knockout for β-arrestin 1 (β1KO), β-arrestin 2 (β2KO) and both (β1/2KO) (A,B) and MEF and MEF expressing truncated IGF-1R, defective in binding β-arr1 (Δ1245) (C,D) were analysed for effects of CP. (A, C) Cells were incubated in serum free medium for 12 h and were unstimulated (SFM) or stimulated with 100 ng/ml CP or 50 ng/ml IGF-1 for 24 h. Protein lysates were analyzed by WB for IGF-1R and GAPDH as a loading control. Signals were quantified by densitometry, normalized to GAPDH and displayed as a percentage of the IGF-1R at 0 h. Data correspond to the mean±SEM from three independent experiments. (B,D) Cell viability of the CP-treated cells for 48 h, in the absence (SFM) or presence of serum, was evaluated by PrestoBlue reagent. Numbers of viable cells following CP treatment are displayed as percentage of untreated controls. Data correspond to the mean±SEM from three independent experiments.

FIG. 4A-FIG. 4B. β-Arrestin1 enhances CP induced IGF-1R downregulation and inhibition of cell proliferation. (A) Cells transfected with different amounts of plasmid encoding β-arrestin1-flag (β1-flag) as indicated were treated without or with 100 ng/ml CP for 24 h. Protein lysates were analyzed by WB for IGF-1R, β-arrestin 1 (β-arr1) and GAPDH as a loading control. Signals were quantified by densitometry, normalized to GAPDH and expressed as percentage of mock transfected, unstimulated controls. Data correspond to the mean±SEM from three independent experiments. (B) Cells transfected as in (A), were treated with 100 ng/ml CP for 48 h. Number of viable cells is displayed as percentage of mock transfected, unstimulated control. Data correspond to the mean±SEM from three independent experiments.

FIG. 5A-FIG. 5B. CP-induced β-arrestin1-mediated IGF-1R signaling activation (A) Cells were incubated in serum free medium for 12 h and then treated with either 50 ng/ml IGF-1 or 100 ng/ml CP for 0, 2, 5, 10, 30 or 60 min. Protein lysates were analyzed by WB for phosphorylated ERK (P-ERK), phosphorylated AKT (P-AKT), phosphorylated IGF-1R (P-IGF-1R) and GAPDH as a loading control, (B) MEF and MEF knockout for β-arrestin1 (β1KO), β-arrestin2 (β2KO) and both (β1/2KO) or MEF with truncated IGF-1R (Δ1245) were incubated in serum free medium for 12 h and then unstimulated (SFM) or stimulated with either 50 ng/ml IGF-1 or 100 ng/ml CP for 10 min. Protein lysates were analyzed by WB for phosphorylated ERK (P-ERK) and GAPDH as a loading control. Signals were quantified by densitometry, normalized to GAPDH and expressed as percentage of ERK activation relative to WT MEF. Data correspond to the mean±SEM from three independent experiments.

FIG. 6A-FIG. 6C. Effect of β-arrestin1 shRNA and MAPK/ERK pathway inhibitor (mitogen activated protein kinase kinase (MEK) inhibitor, U0126) on CP induced cell viability reduction. (A) Indicated cells were stably transfected with doxycycline inducible β-arrestin1 shRNA and treated with doxycycline for four days. The cell viability was measured by PrestoBlue reagent and displayed as a percentage of doxycycline untreated cells. Cell lysates were prepared from the same samples and analyzed by WB for β-arrestin1 (βarr1) and GAPDH as a loading control. Signals were quantified by densitometry, normalized to GAPDH and presented as a percentage of the doxycycline untreated cells. Data correspond to the mean±SEM from three independent experiments. (B) Cells prepared as in (A), were treated without or with 100 ng/ml CP for 48 h and the cell viability assayed by PrestoBlue reagent. The inhibition ratio (quotient between CP-treated and CP-untreated cells) was calculated for each doxycycline dose and displayed as percentage of CP untreated cells. Data correspond to the mean±SEM from three independent experiments. During the experiment, cell lysates were collected at 10 min after CP stimulation and analyzed by WB for P-ERK and total ERK as a loading control and at 12 h after CP stimulation and analyzed by WB for IGF-1R and GAPDH as a loading control. Signals were quantified by densitometry, normalized to the loading control and presented as a percentage of the CP untreated cells. Data correspond to the mean±SEM from three independent experiments. (C) Indicated cells were pre-treated for 60 min without or with the ERK inhibitor U0126, stimulated without or with 100 ng/ml CP. The cell viability assayed 48 h after CP treatment is displayed relative to untreated control. Data correspond to the mean±SEM from three independent experiments. The percentage inhibition of CP treated is displayed relative to CP untreated cells.

FIG. 7. Abolition of CP-induced ERK activation by MAPK/ERK pathway inhibitor (MEK inhibitor, U0126). Cells were incubated in serum-free medium for 12 h and then with MEK) inhibitor U0126 for 60 min before treatment without and with 100 ng/μl CP for 10 min. Protein lysates were analyzed by WB for P-ERK and GAPDH as a loading control.

### DETAILED DESCRIPTION

Owing to its identified role in cancer, insulin-like growth factor type 1 receptor (IGF-1R) targeted therapy is an exciting approach for cancer treatment. IGF-1R antagonist has been regarded as a potential cancer therapy, based on the idea that treatment with IGF-1R antagonists would eventually lead to death of the tumor cell. Yet, when translated into clinical trials, IGF-1R specific antibodies do not fulfill expectations.

Despite significant hopes regarding the use of anti-IGF-1R for cancer treatment, the outcomes of Phase III clinical trials have been rather disappointing. For example, in the case of Ewing's sarcoma, several clinical trials were stopped due to apparent lack if efficacy and some pharmaceutical companies are aborting their programs for developing IGF-1R antagonists for use in cancer therapies. Moreover, based on the clinical trial results with targeting antibodies, the value of IGF-1R as a target for cancer therapy is currently questioned.

The present invention discloses a surprising and practical therapeutic strategy to enhance the response of a subject to anti-IGF-1R therapies for all types of cancer, including childhood sarcoma, melanoma, colon cancer, breast cancer, lung cancer, prostate cancer, pancreatic cancer as well as liver metastasis of any cancer type. Disclosed herein are also compositions for use in such cancer treatment.

As used in the description of the invention and the appended claims, the singular forms “a,” “an” and “the” are intended to include the plural forms as well, unless the context clearly indicates otherwise. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a compound) and the like, is meant to encompass variations of ±20%, ±10%, ±5%, ±1%, ±0.5%, or even ±0.1% of the specified amount.

The terms “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” when used in a claim or the description of this invention is not intended to be interpreted to be equivalent to “comprising.”

As used herein, the terms “increase,” “increases,” “increased,” “increasing,” “enhance”, “enhances”, “enhanced, “enhancing,” “improve,” “improves,” “improved,” “improving,” and similar terms indicate an elevation of at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 400%, 500% and the like or more.

As used herein, the terms “reduce,” “reduces,” “reduced,” “reduction,” and similar terms mean a decrease of at least about 5%, 10%, 15%, 20%, 25%, 35%, 50%, 75%, 80%, 85%, 90%, 95%, 97% or more. In particular embodiments, the reduction results in no or essentially no (i.e., an insignificant amount, e.g., less than about 10% or even 5%) detectable activity or amount.

The term “IGF-1R antagonist” refers to any molecule that blocks, suppresses, inhibits, or reduces a biological activity of IGF-1R, including downstream pathways mediated by IGF-1R signaling. The term “antagonist” implies no specific mechanism of biological action whatsoever, and expressly includes and encompasses all possible pharmacological, physiological, and biochemical interactions with IGF-1R. Examples of an IGF-1R antagonist include (1) an agent that is a competitive inhibitor or a noncompetitive inhibitor of IGF-1R binding to its ligands, thus interfering with the binding of an IGF-1R with its ligands; (2) an agent that does not interfere with the binding of the IGF-1R with its natural ligand but, instead, enhances, modifies, inhibits or decreases the activities caused by binding of the IGF-1R to its ligands; (3) an agent that decreases the expression of IGF-1R. Examples of such agents include antibodies (including antigen binding portions thereof), nucleic acid molecules (such as anti-sense or interfering RNA molecules and aptamers), peptides, non-peptide small organic molecules, and so on.

The term “MAPK/ERK (mitogen-activated protein kinase/extracellular signal-regulated kinase) pathway inhibitor” is used herein to refer to an agent that inhibits the expression level, subcellular distribution and/or activity of an ERK1/2 (Extracellular signal-regulated kinases). The MAPK/ERK pathway has also been called the ERK pathway (Extracellular signal-regulated kinases pathway), the MAPK pathway (Mitogen-activated protein kinases pathway and the Ras-Raf-MEK-ERK pathway. The components of the pathway include: (1) Ras (Rat sarcoma) having the exemplary members of H-Ras, N-Ras and K-ras. Ras is mutated in about 20-30% of all cancers. Activated Ras in turn activates Raf; (2) Raf (Rapidly Accelerated Fibrosarcoma) having the exemplary members of A-Raf, B-Raf and C-Raf (Raf-1). Raf is activated by Ras or by mutation. Activated Raf in turn activates MEK; (3) MEK (Mitogen-activated protein kinase kinase, also known as MAP2K) having the exemplary members of Mek1 (MAP2K1) and Mek 2(MAP2K2). Mek activates ERK; and (4) ERK (extracellular signal-regulated kinases) (also known as MAPK) having the exemplary members of ERK1 (gene MAPK3) and ERK2 (gene MAPK1).

The term “MAPK/ERK inhibitor” encompasses any agent that inhibits MAPK/ERK signaling through its effectors in the RAS-RAF-MEK-ERK signal transduction pathway and any mutants and variants thereof. In some embodiments, a suitable MAPK/ERK inhibitor is one that selectively inhibits MEK (mitogen activated protein kinase kinase MAPKK) activity, e.g., the MAPK/ERK inhibitor selectively inhibits phosphorylation of an ERK polypeptide by a MEK polypeptide, where “selective inhibition” means that the inhibitor does not substantially inhibit an activity of a polypeptide other than a MEK polypeptide (e.g., MEK1 and MEK2 inhibitors). Thus, in some particular embodiments, the term “MAPK/ERK inhibitor” includes, but is not limited to, (1) MEK (e.g., Mek1 and Mek2) inhibitors; (2) Ras (e.g., HRAS, KRAS, NRAS) inhibitors; (3) Raf (e.g., A-Raf, B-Raf, C-Raf) inhibitor; (4) ERK (ERK1 and ERK2) inhibitors; any mutants and variants thereof, and any combination thereof.

“Inhibit,” “inhibited,” and “inhibition” as used herein refers to the use of a MAPK/ERK pathway inhibitor for reducing or inhibiting the IGF-1R signalling activated by an IGF-1R inhibitor, thereby reducing the protective effect of IGF-1R inhibitors on cancer cell viability and increasing the effectiveness of IGF-1R antagonists in killing cancer cells.

By the term “treat,” “treating,” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the progression of the disease or disorder.

The term “treating cancer” or “treatment of cancer” refers to causing a desirable or beneficial effect in a patient diagnosed with a cancer. The desirable or beneficial effect may include: (1) inhibition of further growth or spread of cancer cells, (2) death of cancer cells, (3) inhibition of reoccurrence of cancer, (4) alleviation, reduction, mitigation, inhibition, or reducing the frequency, of symptoms associated with the cancer (such as pain), or (5) improved survival of the patient. Inhibition of reoccurrence of cancer includes inhibition of cancer growth at initial cancer sites and surrounding tissue that have previously been treated by radiation, chemotherapy, surgery, or other techniques, as well as absence of cancer growth at new distant sites. The desirable or beneficial effect can be either patientive or objective. For example, if the patient is human, the human may note improved vigor or vitality or decreased pain as patientive symptoms of improvement or response to therapy. Alternatively, the clinician may notice a decrease in tumor size or tumor burden based on physical exam, laboratory parameters, tumor markers or radiographic findings. Some laboratory signs that the clinician may observe for response to treatment include normalization of tests, such as white blood cell count, red blood cell count, platelet count, erythrocyte sedimentation rate, and various enzyme levels. Additionally, the clinician may observe a decrease in a detectable tumor marker. Alternatively, other tests can be used to evaluate objective improvement, such as sonograms, nuclear magnetic resonance testing and positron emissions testing.

“Effective amount” as used herein refers to an amount of a compound, composition and/or formulation of the invention that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of skill in the art by reference to the pertinent texts and literature and/or by using routine experimentation.

A “treatment effective” amount as used herein is an amount that is sufficient to treat (as defined herein) the subject. Those skilled in the art will appreciate that the therapeutic effects need not be complete or curative, as long as some benefit is provided to the subject.

The term “IGF-1R antagonist and MAPK/ERK pathway inhibitor combination therapy” refers to the administration of an IGF-1R antagonist and a MAPK/ERK pathway inhibitor to a patient for the management of a medical condition.

The term “pharmaceutical composition” as used herein refers to a chemical compound or composition capable of inducing a desired therapeutic effect when administered to a patient (e.g., subject in need thereof) in an effective amount for treating a particular condition (e.g., cancer).

“Pharmaceutically acceptable” refers to those properties or substances that are acceptable to the patient from a pharmacological or toxicological point of view, or to the manufacturing pharmaceutical chemist from a physical or chemical point of view regarding composition, formulation, stability, patient acceptance, bioavailability and compatibility with other ingredients.

“Pharmaceutically acceptable excipient” can mean any substance, not itself a therapeutic agent, used as a carrier, diluent, binder, or vehicle for delivery of a therapeutic agent to a subject, or added to a pharmaceutical composition to improve its handling or storage properties or to permit or facilitate formation of a compound or composition into a unit dosage form for administration. Pharmaceutically acceptable excipients are well known in the pharmaceutical arts and are described, for example, in Remington's Pharmaceutical Sciences, Mack Publishing Co., Easton, Pa. (e.g., 20^{th} Ed., 2000), and Handbook of Pharmaceutical Excipients, American Pharmaceutical Association, Washington, D.C., (e.g., 1^{st}, 2^{nd} and 3^{rd} Eds., 1986, 1994 and 2000, respectively). Excipients may provide a variety of functions and may be described as wetting agents, buffering agents, suspending agents, lubricating agents, emulsifiers, disintegrants, absorbents, preservatives, surfactants, colorants, flavorants, and sweeteners. Examples of pharmaceutically acceptable excipients include without limitation: (1) sugars, such as lactose, glucose and sucrose; (2) starches, such as corn starch and potato starch; (3) cellulose and its derivatives, such as sodium carboxymethyl cellulose, ethyl cellulose, cellulose acetate, hydroxypropylmethylcellulose, and hydroxypropylcellulose; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates or polyanhydrides; and (22) other non-toxic compatible substances employed in pharmaceutical formulations.

“Pharmaceutically acceptable carrier” as used herein refers to a nontoxic carrier or vehicle that does not destroy the pharmacological activity of the compound with which it is formulated. Pharmaceutically acceptable carriers or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, cyclodextrins, sodium carboxymethylcellulose, polyacrylates, waxes, polyethylene-polyoxypropylene-block polymers, polyethylene glycol and wool fat.

“A “subject” or “patient”, as used herein, includes any animal that has or is suspected of having cancer. Such a subject is generally a mammalian subject (e.g., a laboratory animal such as a rat, mouse, guinea pig, rabbit, primate, etc.), a farm or commercial animal (e.g., a cow, horse, goat, donkey, sheep, etc.), or a domestic animal (e.g., cat, dog, ferret, etc.). In particular embodiments, the subject is a primate subject, a non-human primate subject (e.g., a chimpanzee, baboon, monkey, gorilla, eta) or a human. In certain embodiments, a subject of the invention can be a subject known to have or believed to have cancer. A subject of the invention can be a subject known or believed to be at risk of developing cancer. Alternatively, a subject according to the invention can also include a subject not previously known or suspected to have cancer. Subjects include males and/or females of any age, including neonates, juvenile, mature and geriatric subjects, “Subject” can also refer to a cell or tissue, in vitro or in vivo, of an animal or a human.

A “subject in need” of the methods of the invention can be a subject known to have, suspected of having, or having an increased risk of developing cancer.

The inventors have surprisingly found that IGF-1R antagonists, which also downregulate IGF-1 receptors thereby stimulating the MAPK/ERK pathway and thus stimulating ERK signaling. Via this stimulation, IGF-1R treatment actually helps the cancer cells survive. Accordingly, it is an object of the present invention to enhance treatment of cancer using IGF-1R antagonist therapy by administering an IGF-1R antagonist with at least one MAPK/ERK pathway inhibitor in order to counteract the ERK signaling stimulation caused by the IGF-1R antagonist.

This therapy can be used for patients having cancer cells expressing IGF-1R, and this IGF-1R expression may be detected by any means known in the art for detecting the expression of a protein. These methods include but is not limited to detection of IGF-1R protein expression (immunohistochemistry, Western blot, chromatography, protein sequencing, ELISA) detection of IGF1R transcript or IGF1R gene expression (PCR, sequencing, FISH). This therapy can be used for any patient having cancer cells expressing IGF-1R regardless of the activating mutations within the RAS-RAF-MEK-ERK signaling pathway. The treatment of the present invention can be a “one hit treatment” where one or more MAPK/ERK pathway inhibitors are administered simultaneously with one or more IGF-1R antagonists in order to block the negative side effect of the IGF-1R antagonist, i.e. blocking the stimulation of the RAS-RAF-MEK-ERK pathway. In other embodiments, the MAPK/ERK pathway inhibitor can be administered prior to administration of the IGF-1R antagonist.

Any IGF-1R antagonist that induces receptor down regulation may be used according to the present invention Possible IGF-1R antagonists include but are not limited to those provided in Table 1. IGF-1R antagonists downregulating the receptor, activate the RAS-RAF-MEK-ERK pathway and thus activate ERK signaling. Thus, according to the present invention the IGF-1R antagonist can be combined with a MAPK/ERK pathway inhibitor to reduce MAPK/ERK pathway signaling. Any MAPK/ERK pathway inhibitor may be used according to the present invention. Possible MAPK/ERK pathway inhibitors include, but are not limited to, those provided in Table 2.

TABLE 1
Exemplary IGF-1R antagonists.

| IGF-1R antagonist | class | company |
|---|---|---|
| IMC-A12 | fully human IgG1 mAb | ImClone (New York) |
| cixutumumab | against IGF-1R | |
| R1507 | fully human mAb | Genmab (Copenhagen)/Roche |
| | against IGF-1R | (Basel) |
| MK-0646 | humanized IgG1 | Merck |
| | antibody against IGF- | |
| | 1R extracellular | |
| AMG 479 | fully human mAb | Amgen |
| | targeting IGF-1R | |
| AVE1642 | humanized IgG1 mAb | Sanofi-Avensis |
| | against IGF-1R | |
| CP-751,871 | | Pfizer |
| figitumumab | | |
| Tamoxifen | ER inhibitor | |
| Letrozole | Aromatase inhibitor | |
| Metformin | AMP-activated | |
| | protein kinase | |
| | (AMPK) inhibitor | |
| Picropodophyllin | Small molecule tyrosine | Axelar |
| (PPP) | kinase inhibitor (TKI) | |
| BMS-754807 | Small molecule | Bristol-Myers Squibb |
| | TKI | |

TABLE 2
Exemplary MAPK/ERK inhibitors.

| Inhibitor | Type of inhibitor | Target(s) | Company |
|---|---|---|---|
| Tipifarnib | Ras inhibitor | Ras, farnesyl- | Johnson & Johnson |
| (Zarnestra ™, | | transferase, | |
| R115777) | | Rheb | |
| BAY 43-9006 | Raf inhibitor | Raf, VEGFR2, | Bayer |
| (Nexavare ®, | | VEGFR3, | |
| Sorafenib tosylate) | | PDGF-R, | |
| | | c-Kit, c-Fms, | |
| | | Flt-3 | |
| AAL-881 | Raf inhibitor | Raf | Novartis |
| LBT-613 | Raf inhibitor | Raf | Novartis |
| RAF265 | Raf inhibitor | B-Raf, Raf-1 | Novartis |
| | | (c-Raf), | |
| | | A-Raf, | |
| | | B-Raf^{V600E}, | |
| | | VEGFR-2 | |
| XL281 | Raf inhibitor | B-Raf, c-Raf, B- | Exelixis/Bristol |
| | | Raf^{V600E} | Myers Squibb |
| SB-590885 | Raf inhibitor | Raf, B-Raf^{V600E} | GlaxoSmithKline |
| PLX-4720 | Raf inhibitor | Raf, B-Raf^{V600E} | Plexxikon/Roche |
| PLX-4032 | Raf inhibitor | Raf, B-Raf^{V600E} | Plexxikon/Roche |
| L-779,450 | Raf inhibitor | Raf | Merck |
| GW5074 | Raf inhibitor | Raf-1 (c-Raf) | GlaxoSmithKline |
| SB-699393 | Raf inhibitor | Raf | GlaxoSmithKline |
| CI-1040 | MEK inhibitor | MEK1, MKK5 | Pfizer |
| (PD-184352) | | | |
| PD0325901 | MEK inhibitor | MEK1/2 | Pfizer |
| XL518 | MEK inhibitor | MEK | Exelixis |
| Selumetinib | MEK inhibitor | MEK | Astra Zeneca/Array |
| (AZD6244, | | | BioPharma |
| ARRY-142886) | | | |
| RDEA119 | MEK inhibitor | MAP2K1 | Ardea/Bayer |
| (BAY 869766) | | (MAPK/ERK | |
| | | kinase 1) | |
| PD098059 | MEK inhibitor | MEK1/2 | Parke-Davis/Pfizer |
| U0126 | MEK inhibitor | MEK1/2 | DuPont |
| | | | Pharmaceuticals |
| SL-327 | MEK inhibitor | MEK1/2 | DuPont |
| | | | Pharmaceuticals |

Combinations of any IGF-1R antagonist and any MAPK/ERK inhibitor may be used according to the present invention. In representative embodiments, an antibody directed against IGF-1R can be administered in combination with at least one MEK inhibitor (e.g., U0126, PD098059, SL-327 and PD0325901). In other embodiments, a small molecule TKI can be administered in combination with at least one MEK or Raf (e.g., B-Raf) inhibitor. In still other embodiments, an IGF-1R TKI (e.g. BMS-754807) can be administered in combination with an estrogen receptor (ER) inhibitor (Tamoxifen or Letrozol) and a MEK inhibitor (U0126, PD098059, SL-327 and PD0325901).

Accordingly, in one embodiment, the present invention provides a composition comprising at least one insulin growth factor 1 receptor (IGF-1R) antagonist and at least one mitogen activated protein kinase/extracellular signal-regulated kinase (MAPK/ERK) pathway inhibitor.

In an additional aspect, the present invention provides a pharmaceutical composition comprising at least one insulin growth factor 1 receptor (IGF-1R) antagonist and at least one mitogen activated protein kinase/extracellular signal-regulated kinase (MAPK/ERK) pathway inhibitor as described herein and a pharmaceutically acceptable excipient.

In additional aspects of the invention, the composition comprising at least one insulin growth factor 1 receptor (IGF-1R) antagonist and at least one mitogen activated protein kinase/extracellular signal-regulated kinase (MAPK/ERK) inhibitor can further comprise a pharmaceutically acceptable excipient.

The present invention further provides a method of treating cancer in a subject in need thereof, the method comprising administering to said subject a therapeutically effective amount of at least one insulin growth factor 1 receptor (IGF-1R) antagonist and at least one mitogen activated protein kinase/extracellular signal-regulated kinase (MAPK/ERK) pathway inhibitor.

In some embodiments, the present invention provides the use of at least one insulin growth factor 1 receptor (IGF-1R) antagonist and at least one mitogen activated protein kinase/extracellular signal-regulated kinase (MAPK/ERK) pathway inhibitor for the manufacture of a composition for treating cancer.

In other aspects, the present invention provides an IGF-1R antagonist and a mitogen activated protein kinase/extracellular signal-regulated kinase (MAPK/ERK) pathway inhibitor for the manufacture of a composition for use in treating cancer, comprising: administering to a subject in need thereof (at least one) an IGF-1R antagonist and (at least one) MAPK/ERK pathway inhibitor, wherein the MAPK/ERK pathway inhibitor is administered at substantially the same time with the IGF-1R antagonist, the MAPK/ERK pathway inhibitor is administered prior to the IGF-1R antagonist and/or the MAPK/ERK pathway inhibitor is administered after the IGF-1R antagonist.

In still other embodiments, the present invention provides use of at least one mitogen activated protein kinase/extracellular signal-regulated kinase (MAPK/ERK) inhibitor for the manufacture of a pharmaceutical composition for inhibition of the enhancement of IGF-1R receptor down regulation by an IGF-1R inhibitor.

In some embodiments, an IGF-1R antagonist can be, but is not limited to, an antibody and/or monoclonal antibody (mAb) directed against IGF-1R. In other embodiments, a monoclonal antibody can be a humanized monoclonal antibody. In particular embodiments, the humanized monoclonal antibody can be, but is not limited to, IMC-A12 (cixutumumab), R1507, MK-0646, AMG 479, AVE1642, CP-751,871 (figitumumab), or any combination thereof. In particular embodiments, the IGF-1R antagonist is CP-751,871 (figitumumab).

In other embodiments of the invention, an IGF-1R antagonist can be but is not limited to a small molecule inhibitor in combination with an estrogen receptor (ER) inhibitor, an aromatase inhibitor, and or an AMP kinase (AMPK) inhibitor or any other drug combination that enhances or increases IGF-1R downregulation. In particular embodiments, the small molecule TKI inhibitor can be PPP and/or BMS-754807 and the estrogen receptor inhibitor, and/or aromatase inhibitor can be Tamoxifen and/or Metformin, respectively.

In other embodiments of the invention, a MAPK/ERK pathway inhibitor can be, but is not limited to, a Ras (e.g., products of the HRAS, KRAS and NRAS genes and mutants and variants thereof) (Rat sarcoma) inhibitor, a Raf (Rapidly Accelerated Fibrosarcoma) (e.g., products of the C-Raf, B-Raf and A-Raf genes and mutants and variants, thereof) inhibitor, an ERK (e.g., ERK1, ERK2) (Extracellular Signal-Regulated Kinase) inhibitor and/or a Mitogen-activated protein kinase kinase (also known as MAP2K) (MEK) (e.g., Mek1, Mek2) inhibitor. In particular embodiments, the MAPK/ERK pathway inhibitor is a MEK inhibitor.

In still other embodiments of the invention, a Ras inhibitor can be, but is not limited to, Tipifarnib (Zarnestra™, R115777). In additional embodiments of the invention, a Raf inhibitor can be, but is not limited to BAY 43-9006 (Nexavar®, Sorafenib tosylate), AAL-881, LBT-613, SB-590885, PLX-4720, L-779,450, SB-699393, or any combination thereof.

In other embodiments, a Raf inhibitor can be, but is not limited to BAY 43-9006 (Nexavar®, Sorafenib tosylate), AAL-881, LBT-613, SB-590885, PLX-4720, L-779,450, SB-699393,

In further embodiments of the invention, a Raf inhibitor can be, but is not limited to, RAF265, XL281, PLX-4032, or any combination thereof.

In other embodiments of the invention, a MEK inhibitor can be, but is not limited to, CI-1040 (PD-184352), PD0325901, XL518, Selumetinib (AZD6244, ARRY-142886), RDEA119 (BAY 869766), PD098059, U0126, SL-327, or any combination thereof. In some embodiments, the MEK inhibitor is PD0325901. In other embodiments, the MEK inhibitor is U0126. In still other embodiments, the MEK inhibitor is PD098059.

In some embodiments, the invention provides compositions and methods for treating cancer in a subject in need thereof, wherein the subject in need thereof has a cancer comprising cells that express IGF-1R.

In particular embodiments, the invention provides compositions and methods for treating cancer wherein the cancer can be, but is not limited to, Ewing's sarcoma, childhood sarcoma, colon cancer, melanoma, breast cancer, breast cancer, hepatic cancer, liver metastasis of cancer, liver metastasis of uveal melanoma, colon cancer, prostate cancer, lung cancer, pancreatic cancer, uveal melanoma, ovarian cancer, gastric cancer and any combination thereof.

The at least one insulin growth factor 1 receptor (IGF-1R) antagonist and at least one mitogen activated protein kinase/extracellular signal-regulated kinase (MAPK/ERK) pathway inhibitor can be administered to a patient in need thereof in any order as well as simultaneously or concurrently. Thus, in one embodiment, the MAPK/ERK inhibitor can be administered at substantially the same time with the IGF-1R antagonist. In another embodiment, the MAPK/ERK inhibitor can be administered prior to the IGF-1R antagonist. In a further embodiment, the MAPK/ERK inhibitor is administered after the IGF-1R antagonist.

As used herein, the word “concurrently” or “substantially the same time” means sufficiently close in time to produce a combined effect (that is, concurrently can be simultaneously, or it can be two or more events occurring within a short time period before or after each other).

As used herein the term “concomitant administration” or “combination administration” of the therapeutic agents of this invention (e.g., IGF-1R and MAPK/ERK inhibitors) means administration of said agents at such time that each of the therapeutic agents will have a therapeutic effect. In some embodiments, this therapeutic effect will be synergistic. Such concomitant administration can involve concurrent (i.e., at the same time), prior, or subsequent administration of the agents with respect to each other. A person of skill in the art would have no difficulty determining the appropriate timing, sequence and dosages of administration for particular therapeutic agents and compositions of the present invention.

In addition, in some embodiments, the IGF-1R antagonists and MAPK/ERK inhibitors of this invention will be used, either alone or in combination with each other or in combination with one or more other therapeutic medications as described above, or their salts or esters, for manufacturing a medicament for the purpose of providing treatment for cataplexy to a patient or subject in need thereof.

By the term “combination” is meant either a fixed combination in one dosage unit form, or a kit of parts for the combined administration where IGF-1R antagonists and MAPK/ERK inhibitors may be administered independently, at the same time, or separately within time intervals that especially allow that the combination partners show a cooperative, e.g., additive or synergistic, effect, or any combination thereof.

In some particular embodiments, the MAPK/ERK inhibitor is administered one time at the initiation of cancer treatment in the subject in need thereof. In other embodiments, the MAPK/ERK inhibitor is administered each time an IGF-1R antagonist is administered throughout the course of treatment for cancer.

In some embodiments, when the MAPK/ERK inhibitor is administered prior to the IGF-1R antagonist, the MAPK/ERK inhibitor can be administered in a range from about 1 minute to about 48 hours prior to the administration of the IGF-1R antagonist. In particular embodiments, the MAPK/ERK inhibitor can be administered in a range from about 1 minute to about 60 minutes prior to the administration of the IGF-1R antagonist, about 1 minute to about 2 hours prior to the administration of the IGF-1R antagonist, about 1 minute to about 4 hours prior to the administration of the IGF-1R antagonist, about 1 minute to about 6 hours prior to the administration of the IGF-1R antagonist, from about 1 minute to about 8 hours prior to the administration of the IGF-1R antagonist, from about 1 minute to about 10 hours prior to the administration of the IGF-1R antagonist, from about 1 minute to about 12 hours prior to the administration of the IGF-1R antagonist, from about 1 minute to about 15 hours prior to the administration of the IGF-1R antagonist, from about 1 minute to about 20 hours prior to the administration of the IGF-1R antagonist, from about 1 minute to about 24 hours prior to the administration of the IGF-1R antagonist, from about 1 minute to about 30 hours prior to the administration of the IGF-1R antagonist, from about 1 minute to about 36 hours prior to the administration of the IGF-1R antagonist, from about 1 minute to about 40 hours prior to the administration of the IGF-1R antagonist, from about 1 minute to about 44 hours prior to the administration of the IGF-1R antagonist, and the like.

Thus, in particular embodiments, the MAPK/ERK inhibitor can be administered 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, 30 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, 60 min, 75 min, 90 min, 2 hours, 2.25 hours, 2.5 hours, 2.75 hours, 3 hours, 3.25 hours, 3.5 hours, 3.75 hours, 4 hours, 4.25 hours, 4.5 hours, 4.75 hours, 5 hours, 5.25 hours, 5.5 hours, 5.75 hours, 6 hours, 6.25 hours, 6.5 hours, 6.75 hours, 7 hours, 7.25 hours, 7.5 hours, 7.75 hours, 8 hours, 0.825 hours, 8.5 hours, 8.75 hours, 9 hours, 9.25 hours, 9.5 hours, 9.75 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, and the like, and any range therein, prior to the administration of the IGF-1R antagonist.

In other embodiments, when the MAPK/ERK inhibitor is administered after the IGF-1R antagonist, the MAPK/ERK inhibitor can be administered in a range from about 1 minute to about 48 hours after the administration of the IGF-1R antagonist. In particular embodiments, the MAPK/ERK inhibitor can be administered in a range from about 1 minute to about 60 minutes after the administration of the IGF-1R antagonist, about 1 minute to about 2 hours after the administration of the IGF-1R antagonist, about 1 minute to about 4 hours after the administration of the IGF-1R antagonist, about 1 minute to about 6 hours after the administration of the IGF-1R antagonist, from about 1 minute to about 8 hours after the administration of the IGF-1R antagonist, from about 1 minute to about 10 hours after the administration of the IGF-1R antagonist, from about 1 minute to about 12 hours after the administration of the IGF-1R antagonist, from about 1 minute to about 15 hours after the administration of the IGF-1R antagonist, from about 1 minute to about 20 hours after the administration of the IGF-1R antagonist, from about 1 minute to about 24 hours after the administration of the IGF-1R antagonist, from about 1 minute to about 30 hours after the administration of the IGF-1R antagonist, from about 1 minute to about 36 hours after the administration of the IGF-1R antagonist, from about 1 minute to about 40 hours after the administration of the IGF-1R antagonist, from about 1 minute to about 44 hours after the administration of the IGF-1R antagonist, and the like.

Thus, in particular embodiments, the MAPK/ERK inhibitor can be administered 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, 30 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, 60 min, 75 min, 90 min, 2 hours, 2.25 hours, 2.5 hours, 2.75 hours, 3 hours, 3.25 hours, 3.5 hours, 3.75 hours, 4 hours, 4.25 hours, 4.5 hours, 4.75 hours, 5 hours, 5.25 hours, 5.5 hours, 5.75 hours, 6 hours, 6.25 hours, 6.5 hours, 6.75 hours, 7 hours, 7.25 hours, 7.5 hours, 7.75 hours, 8 hours, 0.825 hours, 8.5 hours, 8.75 hours, 9 hours, 9.25 hours, 9.5 hours, 9.75 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, and the like, and any range therein, after administration of the IGF-1R antagonist.

Therefore, in some embodiments, the MAPK/ERK pathway inhibitor and the IGF-1R antagonist can be administered in a single unit dosage form. In other embodiments, the MAPK/ERK pathway inhibitor and the IGF-1R antagonist can be administered in multiple, separate unit dosage forms.

In addition, in some embodiments, the IGF-1R antagonists and the MAPK/ERK pathway inhibitors can be used, either alone and/or in combination with each other and/or in combination with one or more other therapeutic medications as described above, or their salts or esters, for manufacturing a medicament for the purpose of providing treatment for cancer to a patient or subject in need thereof.

A person of skill in the art will be able without undue experimentation, having regard to that skill and this disclosure, to determine a therapeutically effective amount of a IGF-1R antagonist, a MAPK/ERK pathway inhibitor, and any other active agent of this invention (e.g., small molecule TKI inhibitors, ER inhibitors and the like) for the practice of this invention (see, e.g., Lieberman, Pharmaceutical Dosage Forms (Vols. 1-3, 1992); Lloyd, 1999, The Art, Science and Technology of Pharmaceutical Compounding; and Pickar, 1999, Dosage Calculations). A therapeutically effective dose is also one in which any toxic or detrimental side effects of the active agent is outweighed in clinical terms by therapeutically beneficial effects. It is to be further noted that for each particular subject, specific dosage regimens should be evaluated and adjusted over time according to the individual need and professional judgment of the person administering or supervising the administration of the MAPK/ERK inhibitors and the IGF-1R antagonists.

In this context, a therapeutically effective dosage of the biologically active agent(s) (e.g., MAPK/ERK pathway inhibitors, IGF-1R antagonists) can include repeated doses within a prolonged treatment regimen that will yield clinically significant results to provide treatment for cancer. Determination of effective dosages in this context is typically based on animal model studies followed up by human clinical trials and is guided by determining effective dosages and administration protocols that significantly reduce the occurrence or severity of targeted exposure symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays).

Using such models, only ordinary calculations and adjustments are typically required to determine an appropriate concentration and dose to administer a therapeutically effective amount of the biologically active agent(s) (e.g., amounts that are orally effective intranasally effective, transdermally effective, intravenously effective, or intramuscularly effective to elicit a desired response). The effective amount, however, may be varied depending upon the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

In an exemplary embodiment of the present invention, unit dosage forms of the compositions of IGF-1R antagonists and the MAPK/ERK pathway inhibitors are prepared for standard administration regimens. In this way, the composition(s) can be subdivided readily into smaller doses at the physician's direction. For example, unit dosages can be made up in packeted powders, vials or ampoules.

Thus, as appropriate, a treatment effective amount in any individual case can be determined by one of ordinary skill in the art by reference to the pertinent texts and literature and/or by using routine experimentation (see, e.g., Remington, The Science and Practice of Pharmacy (21^{st} ed. 2005)). In one embodiment, a IGF-1R antagonist, a MAPK/ERK pathway inhibitor, and any other active agent of this invention is administered at a dose of about 0.001 to about 10 mg/kg body weight, e.g., about 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9, or 10 mg/kg. In some instances, the dose can be even lower, e.g., as low as 0.0005 or 0.0001 mg/kg or lower. In some instances, the dose can be even higher, e.g., as high as 20, 50, 100, 500, or 1000 mg/kg or higher. The present invention encompasses every sub-range within the cited ranges and amounts.

In representative embodiments, effective administration of the compositions of this invention can be, at an oral or parenteral dose of from about 1 mg/kg/dose to about 20 mg/kg/dose for the IGF-1R antagonist (e.g., 1, 2, 3, 4, 5, 6, 7, 8, 9, 10, 11, 12, 13, 14, 15, 16, 17, 18, 19, 20 mg/kg/dose, and the like, and any range therein) and the administration regime can be, for example, from about 1 mg twice daily (BID)/dose to about 100 mg twice daily (BID)/dose for the MAPK/ERK pathway inhibitors (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 mg/kg/dose, and the like, and any range therein). Therefore, the therapeutically effective amount of the active ingredient can be, for example, from about 70 mg/three weeks, in one dose to about 1400 mg/three weeks in one dose or any range therein for a subject having, for example, an average weight of 70 kg, for the IGF-1R antagonist and from about 1 mg/day to about 50 mg/day for the MAPK/ERK pathway inhibitor or any range therein.

The IGF-1R antagonists and MAPK/ERK pathway inhibitors and other active agents of this invention may be administered by any medically appropriate procedure (e.g., oral, rectal, buccal (e.g., sub-lingual), vaginal, parenteral (e.g., subcutaneous, intramuscular, intradermal, or intravenous), topical (i.e., both skin and mucosal surfaces, including airway surfaces) and transdermal administration.) In representative embodiments, the agents of this invention are delivered to the subject via oral, rectal, buccal (e.g., sub-lingual), vaginal, parenteral (e.g., subcutaneous, intramuscular, intradermal, or intravenous), topical (i.e., both skin and mucosal surfaces, including airway surfaces) and transdermal methods of administration). The particular method of administration chosen will depend on the agent being delivered, the combination of agents to be delivered as well as the particular condition being treated.

The methods of this invention also provide for kits for use in providing treatment for cancer. After a pharmaceutical composition comprising one or more IGF-1R antagonist and one or more MAPK/ERK pathway inhibitor, it can be placed in an appropriate container and labeled for providing treatment for cancer. Alternatively, each of the therapeutic compounds/agents (e.g., IGF-1R antagonist, MAPK/ERK pathway inhibitor) can be placed in the suitable container as individual pharmaceutical compositions. Additionally, another pharmaceutical comprising at least one other therapeutic agent can be placed in the container as well and labeled for treatment of the indicated disease. Such labeling can include, for example, instructions concerning the amount, frequency and method of administration of each pharmaceutical.

### EXAMPLE 1

### Reagents

Monoclonal antibodies against ubiquitin (P4D1), GRK2 (C-9), GRK6 (XX-4) and polyclonal antibodies against IGF-1R (H-60), β-arrestin1 (K-16) and GAPDH (FL-335) were from Santa Cruz Biotechnology Inc. Monoclonal β-arr1 antibody was from BD biosciences (Santa Cruz, Calif., USA). Polyclonal antibodies against phosphorylated (P−)AKT (S473), AKT, P-ERK1/2, ERK1/2, P-IGF1R and IGF-1R were from Cell Signaling Technology (Danvers, Mass., USA).

Cell Culture

Ewing's sarcoma cell lines used in this study are commercially available (SKES, RDES, CADO and A673). The Ewing's sarcoma cell lines LAP35, SKNMC and breast cancer cell line SKBR3 were from Dr Katia Scotlandi (Istituto Ortopedico Rizzoli di Bologna, Italy [1]. Mouse embryonic fibroblast (MEF), MEF with targeted disruption of the IGF-1R gene (R−) and stable transfection with human IGF-1R gene (R+), Δ1245 cell lines (IGF-1R KO cells, stably transfected with IGF-1R with C terminus truncated at position 1245) were a kind gift from Dr. Renato Baserga (Thomas Jefferson University, Philadelphia, Pa., USA) [2]. MEF knockout for β-arrestin1, β-arrestin2 and knockout of both β-arrestin1 and 2 were kindly provided by Dr. Robert J. Lefkowitz (Duke University Medical Center/Howard Hughes Medical Institute, Durham, N.C.) [3].

Small Interfering RNAs (siRNAs) and Transfection

Chemically synthesized, double stranded siRNAs were purchased from Dharmacon, Inc, (Pierce, Ill., USA). The siRNA sequence that was used to deplete endogenous β-arrestin1 was 5′-AAAGCCUUCUGUGCUGAGAAC20-3′ (3). A non-targeting RNA duplex (5′-AAUUCUCCGAACGUGUCACGU-3′) was used as a control. The cells were transfected at 40-50% confluency in 6-well plates using the DharmaFECT 1 transfection reagent (Pierce, Ill.) according to the manufacturer's instructions.

Plasmids and Transfection—

The plasmid expressing β-arrestin1-flag was a kind gift from Dr. Robert J. Lefkowitz (Duke University Medical Center/Howard Hughes Medical Institute, Durham, N.C.). Cells were cultured at 90% confluency in 6-well plates and transfected with plasmids using Lipofectamine 2000 (Invitrogen, CA, USA) according to the manufacturer's instructions.

Generating Inducible β-Arrestin1 shRNA Stable Cell Lines

Stably transfected cell lines were generated for A673, CADO, RDES, SKES and SKNMC Ewing's sarcoma cell lines using Knockout™ Single Vector Inducible RNAi System (Clontech, Mountain View, Calif., USA). Oligonucleotides used: sense: ′5-TCGAGGTCTGGATAAGGAGATCTATTTCAAGAGAATAGATCTCCTTATCCAGATT TTTTACGCGTA-3′ and antisense: 5′-AGCTTACGCGTAAAAAATCTGGATAAGGAGATCTATTCTCTTGAAATAGATCTCC TTATCCAGACC-3′, Sigma Aldrich, St Louis, Mo., USA).

Immunoprecipitation

Cells were lysed with 500 μl lysis buffer (110 mM KOAc, 0.5% (v/v) Triton X-100, 100 mM NaCl, protease inhibitor cocktail tablet (Roche), buffering salts pH 7.4). 500 μg of protein was incubated with Dynabeads protein G (10 μl) (Invitrogen, CA, USA) and 1 μg antibody overnight at 4° C. on a rotator platform. The immunoprecipitates were collected on a magnetic holder, the supernatant discarded, and the beads were washed three times with lysis buffer and then dissolved in the sample buffer for SDS-PAGE.

SDS-PAGE and Western Blotting

Protein samples were dissolved in LDS sample buffer (Invitrogen, CA, USA) and analyzed by SDS-PAGE with 4-12% Bis-Tris gel (Invitrogen, CA, USA). After separation, the proteins were transferred to nitrocellulose membranes at appropriate voltage for 1 h. Membranes were then blocked for 1 h at room temperature in a solution of 5% (w/v) skimmed milk powder and 0.1% (v/v) Tween 20 in Tris-buffered saline (TBS), pH 7.5 (TBS-T). Appropriate primary antibody was incubated overnight at 4° C. Following washing three times in TBS-T, membrane was incubated with a horseradish peroxidase-labeled secondary antibody (Pierce, Ill., USA) for 1 h. The detection was performed with ECL substrate (Pierce, Ill., USA) and exposure to x-ray film.

Cell Viability Assay

Cells were incubated in 96 well tissue culture plates and cell viability was measured by PrestoBlue cell viability assay (Invitrogen, CA, USA) according to manufacturer's instructions. Fluorescence was measured with excitation at 560 nm and emission at 590 nm. Cell number was calculated from standard curves of fluorescence measurements from known numbers of cells. Triplicate cultures were performed on all experiments.

Densitometry Analysis

Band intensity was measured by Quantity One Analysis software (Bio-Rad, CA, USA) and displayed relative to band intensity of the stated loading control.

### Antibodies Targeting IGF-1R Act as Biased IGF-1R Agonists Simultaneously Stimulating ERK Signaling Activation.

### Effects of CP-751,871 Treatment on Cell Viability in Ewing's Sarcoma Cell Lines.

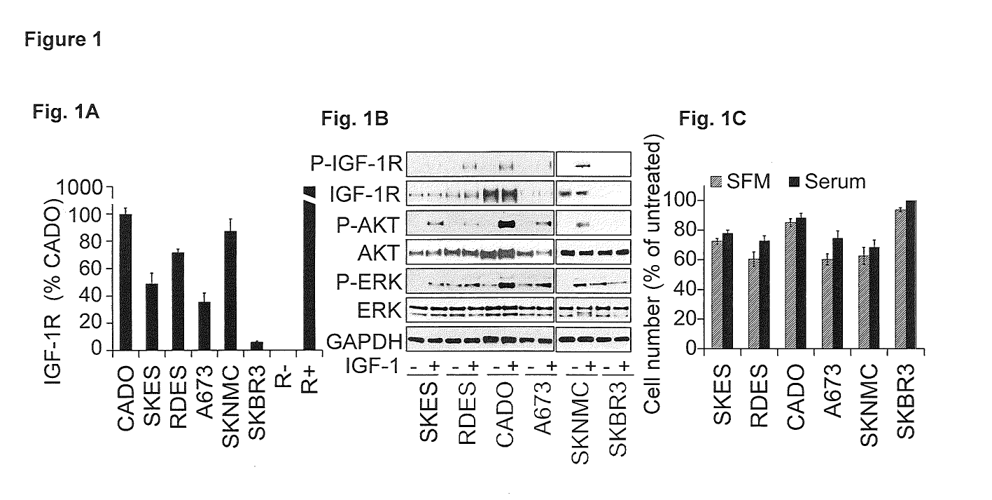

In the first set of experiments we characterized our experimental model regarding the expression of IGF-1R, its capability to mediate IGF-1 signaling as well as its sensitivity to CP-751,871 (Pfizer). IGF-1R expression was investigated in a panel of validated Ewing's sarcoma (ES) cell lines: SKES, RDES, CADO, A673 and SKNMC. Breast cancer cell line SKBR3, expressing low IGF-1R levels and IGF-1R knockout MEF cells (R−) were used as negative controls. R− cells stably transfected with wild type human IGF-1R(R+) were used as a positive control. As shown in FIG. 1A, all the ES cell lines tested expressed IGF-1R. We also tested the function of IGF-1R by analyzing the IGF-1 induced receptor autophosphorylation of the activation loop and the subsequent activation of the two major IGF-1R downstream signaling pathways: MAPK/ERK and PI3K/AKT. Serum starved cells were stimulated with IGF-1 for 10 min and levels of phosphorylated (P−)IGF-1R, P-ERK and P-AKT detected by western blotting analysis (WB), (FIG. 1B). Total IGF-1R, AKT, ERK and GAPDH were used as controls. In all ES cell lines, stimulation with 50 ng/ml IGF-1 induces receptor phosphorylation at tyrosine residues and subsequent signaling activation through the ERK and AKT pathways, demonstrating a functional IGF-1R response. Finally we investigated the sensitivity to antibody targeting IGF-1R by measuring the cell viability of ES cell lines following CP-751,871 treatment. CP-751,871 at 100 ng/ml (CP-751,871 molar concentration 10 fold less compared to 50 ng/ml IGF-1) was added to the cells and the cells incubated in medium in the presence or absence of serum for 48 h. As shown in FIG. 1C, the negative control cells were unaffected whereas all ES cell lines respond to CP-751,871 treatment with decreased cell number Notably, CP-751,871 alone consistently decreases cell proliferation in the absence of serum in all ES cell lines suggesting that inhibitory effects of CP-751,871 on cell proliferation are not essentially dependent on competition with the ligand.

### Mechanism of CP-751,871 Induced IGF-1R Downregulation: β-Arrestin1 Recruitment and Receptor Ubiquitination.

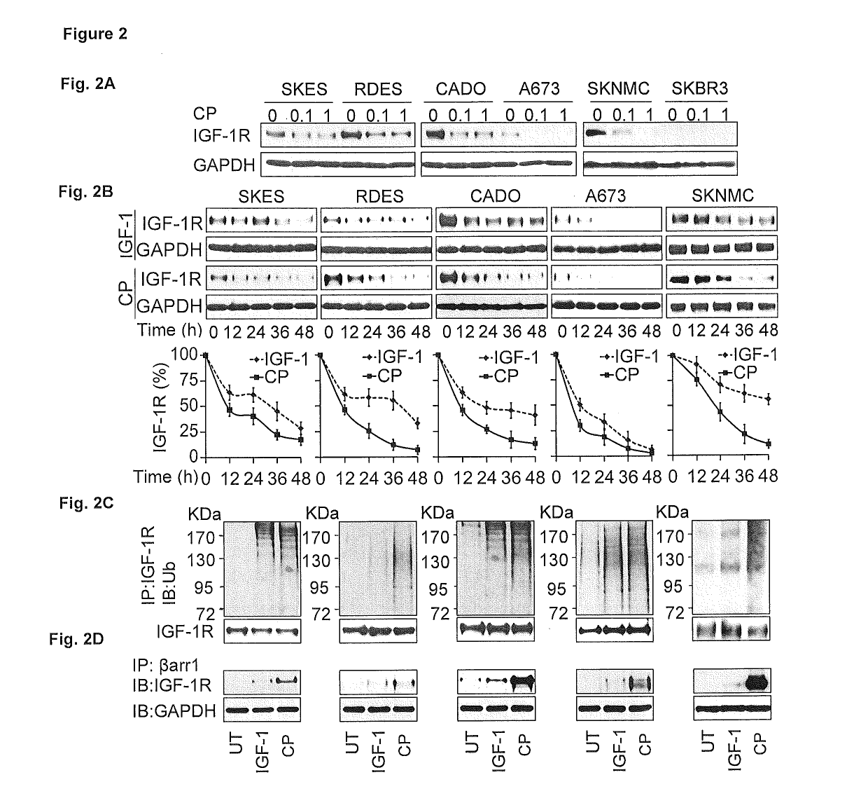

The next experiments were designed to investigate in detail the CP-751,871 effects on IGF-1R downregulation. ES cell lines, serum starved for twelve hours, were treated with CP-751,871 concentrations of 100 ng/ml or 1 μg/ml for 24 h and cell lysates analyzed for IGF-1R expression using GAPDH as a loading control. As shown in FIG. 2A, all ES cell lines downregulate IGF-1R in a dose dependent manner in response to CP-751,871. In a time-response experiment, comparing the CP-751,871 and IGF-1 effects on receptor downregulation, (molar concentration CP-751,871 approximately 10 fold less than IGF-1), CP-751,871 was proven to be more efficient at inducing receptor degradation in four ES cell lines: SKES, RDES, CADO and SKNMC. Similar, very fast, rates of receptor degradation by both C-751,871P and IGF-1 were observed in A673. These trends were confirmed by densitometric quantification of multiple experiments (FIG. 2B, graphs). As the major outcome of ligand binding to the IGF-1R is receptor ubiquitination, “tagging” the receptor for degradation, to identify the mechanism underlying the effects of CP-751,871 on IGF-1R degradation we investigated receptor ubiquitination. The IGF-1R was immunoprecipitated from serum starved ES cells, which had been stimulated, or not for a short time (10 min) with CP-751,871 or IGF-1 and the ubiquitinated receptors detected by WB. Ligand dependent ubiquitination of the IGF-1R was clearly detected and CP-751,871 was more potent in generating receptor ubiquitination in all investigated cell lines (FIG. 2C), consistent with receptor down-regulation experiments (FIG. 2B). Given that β-arrestin1 (β-arr1) is a key protein involved in IGF-1R ubiquitination, we also investigated ligand induced IGF-1R/β-arr1 association. As demonstrated in FIG. 2D, CP-751,871 was much more potent than IGF-1 in recruiting β-arr1 to the receptor in all ES cell lines. Taken together, these experiments demonstrate that CP-751,871 stimulates β-arr1 recruitment to the receptor with subsequent receptor ubiquitination and degradation.

### β-Arrestin1 Dependence of CP Induced IGF-1R Degradation

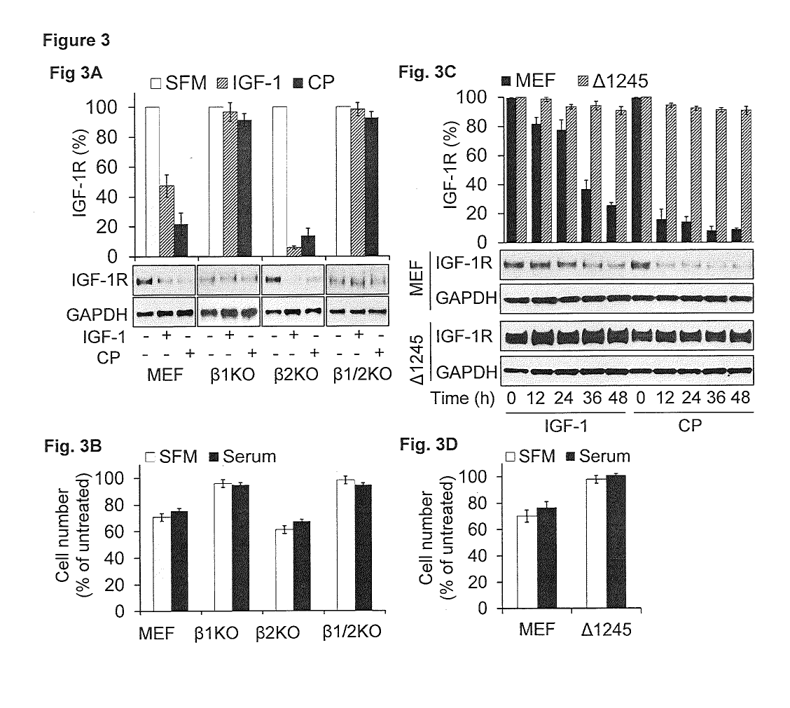

Having shown that CP-751,871 results in β-arr1/IGF-1R association, we next asked whether there is a causative relationship between this process and receptor degradation. To answer this we used MEF cells expressing or not β-arr1(β1KO), β-arr2 or both. Cells were serum starved and the effects of CP-751,871 or IGF-1 stimulation on IGF-1R degradation and cell survival were measured after 24 and 48 h, respectively (FIG. 3A). In control cells, expressing both β-arr isoforms, as well as in the cells expressing only β-arr1 isoform (β2KO), CP-751,871 or IGF-1 treatment drastically downregulated the receptor, whereas in the MEF cells without β-arr1 (β1KO or double knock out, β1/2 KO) this effect was almost completely abolished. This β-arr1 dependency was also displayed in the cell survival experiments: the β1KO or β1/2KO cells were insensitive to CP-751,871 treatment, whereas the corresponding WT or β2KO cells containing β-arr1 responded with a 30-40% inhibition rate, regardless of the presence or absence of serum (FIG. 3B).

Previous data indicates that an IGF-1R truncated at position 1245 (Δ1245) lacks the ability to bind β-arr. To fully validate β-arr1 as a key mediator of CP-751,871 induced IGF-1R downregulation we used an alternative experimental model of MEF cells expressing full length, wild type IGF-1R and MEF cells knock-out for IGF-1R (R−) stably transfected with the C-terminal truncated Δ1245 IGF-1R (FIG. 3C). Over 48 h, the truncated IGF-1R, which is defective in binding β-arr1, was resistant to CP-751,871 or IGF-1 induced degradation whereas full-length IGF-1R, expressed in the same cellular background, displayed a time-dependent degradation rate with CP-751,871 being more efficient than IGF-1, even at a 10 fold lower molar concentration. In line with the results described in the ES models, a decrease in cell number parallels the CP-751,871 induced IGF-1R downregulation, with the MEF cells expressing truncated IGF-1R being essentially unresponsive (FIG. 3D). Taken together, these experiments validate β-arr1 as a key molecule controlling the CP-751,871 induced IGF-1R downregulation.

### β-Arrestin1 Enhances CP-751,871 Induced IGF-1R Downregulation and Inhibition of Cell Proliferation

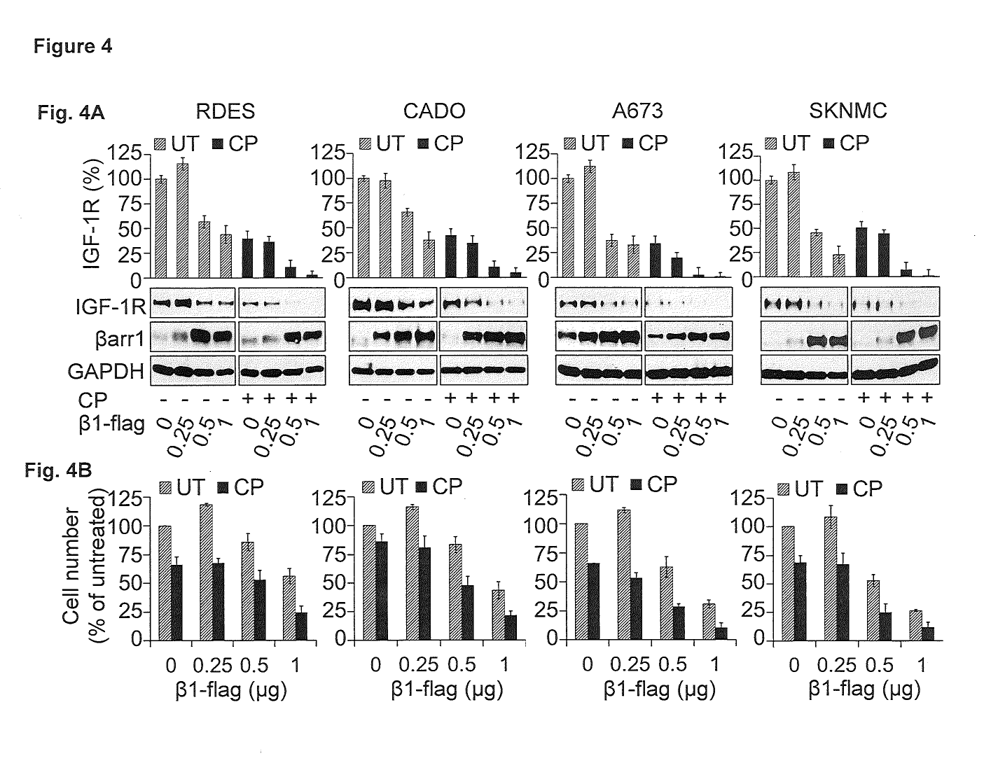

As β-arr1 plays an essential role in CP-751,871 induced IGF-1R downregulation, we next explored whether β-arr1 overexpression could enhance CP-751,871 effects on ES cells, as regards IGF-1R downregulation and overall cell survival. This was done by CP-751,871 treatment of cells transiently transfected with different amounts of β-arr1-flag plasmid. As demonstrated in FIG. 4A, in the absence of the ligand, β-arr1 overexpression down-regulates IGF-1R expression in a dose dependent manner. Nevertheless, increased β-arr1 expression potentiates CP-751,871-induced receptor degradation and enhances the CP-751,871 induced inhibition of cell proliferation/survival (FIG. 4B). Intriguingly, the clear β-arr1 dose-dependent decrease of IGF-1R expression and cell proliferation by CP-751,871 was not observed in cells expressing the lowest amount of exogenous β-arr1, pointing to a possible increased proliferation by CP-751,871 after small increases in β-arr1 level.

### CP-751,871-Induced β-Arrestin1-Mediated ERK Signaling Activation

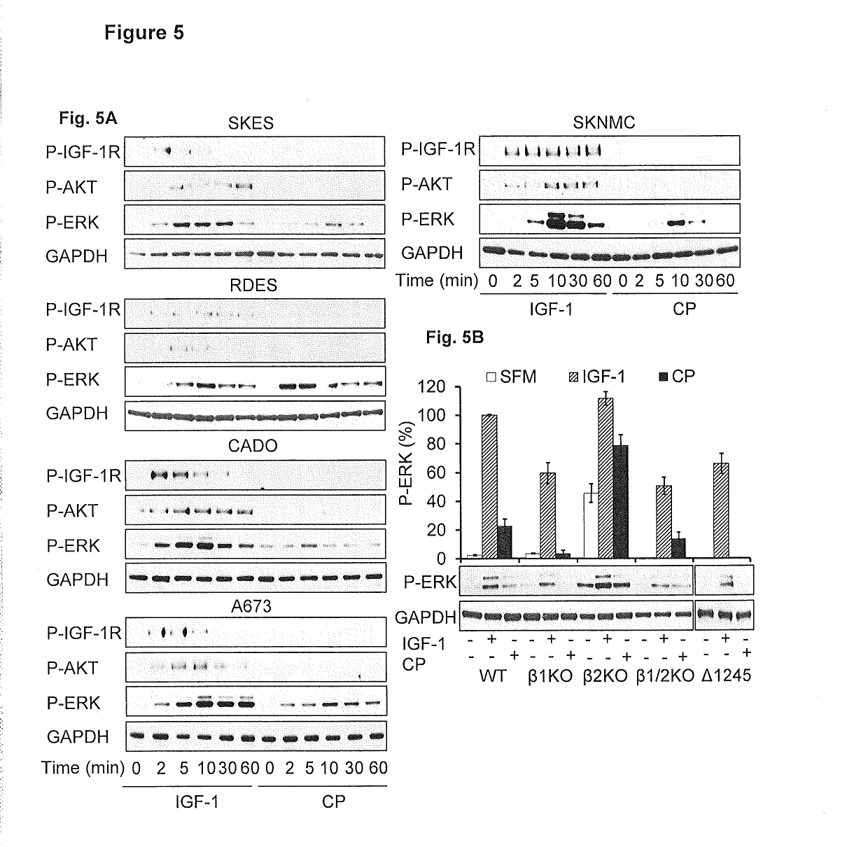

In the next experiments we explored the possible agonistic properties of CP-751,871, secondary to β-arr1 recruitment by investigating the dynamics of IGF-1 or CP-751,871 mediated activation of the two key downstream IGF-1R signaling pathways, the Ras/Raf/MEK/ERK pathway and the PI3K/AKT. Serum starved cells were stimulated with IGF-1 or CP (10:1 molar ratio) for up to 60 min before analyzing by WB. Upon IGF-1 stimulation, the IGF-1R activation loop was phosphorylated within 2 min, demonstrating an increase in its kinase activity. Consequently, both main downstream signaling pathways were activated as demonstrated by ERK and AKT phosphorylation (FIG. 5A). In the case of CP-751,871 stimulation, IGF-1R and AKT phosphorylation were undetectable; however clear ERK phosphorylation signals induced by CP-751,871 were displayed in all ES cell lines. ERK activation levels were generally lower compared to IGF-1 mediated signaling activation, suggesting ERK phosphorylation independent of the IGF-1R kinase activity, possibly through a β-arr mediated mechanism; To confirm this possibility, we again used the MEF cells expressing or not the two β-arr isoforms as well as the MEF expressing the β-arr binding defective IGF-1R. As demonstrated in FIG. 5B, CP-751,871 failed to activate the ERK phosphorylation in the absence of β-arr1, whereas the same pathway was clearly activated in the WT and the β2KO cells. The R− cells expressing truncated IGF-1R, unable to bind β-arr1, were insensitive to CP-751,871 induced ERK activation. Taken together, these experiments demonstrated the partial agonistic properties of CP-751,871, mediated by IGF-1R and β-arr1.

### β-Arrestin1 Dichotomy in Mediating CP-751,871 Effects on Cell Proliferation/Survival: Therapeutic Implications

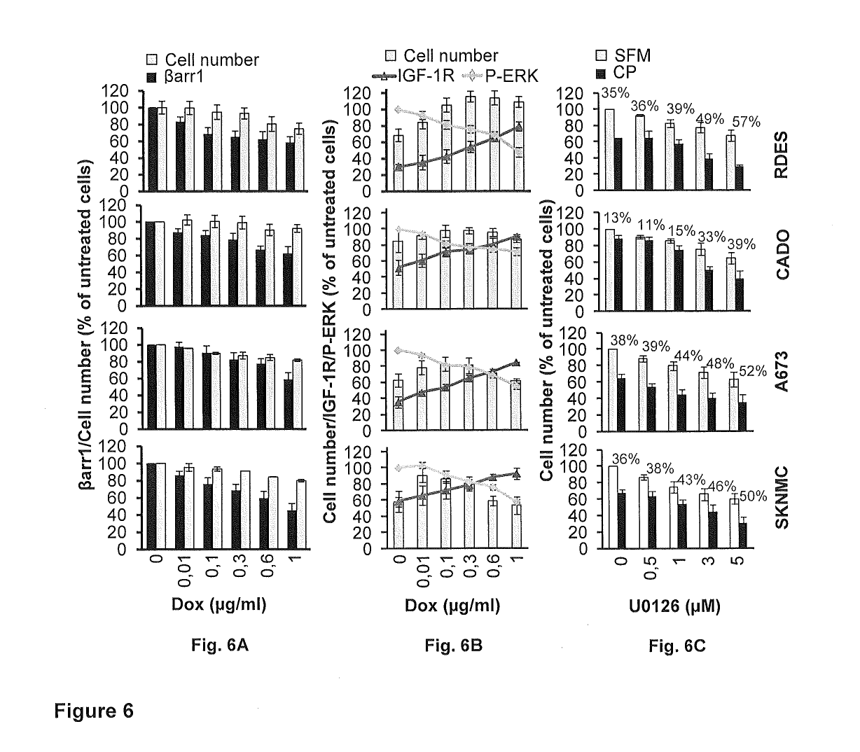

To verify the protective effects of β-arr1 against CP-751,871 induced IGF-1R downregulation and decreased cell survival we performed a rescue experiment by decreasing the β-arr1 levels in our panel of cell lines. The ES cell lines were stably transfected with a doxycycline inducible shRNA β-arr1 knockdown system, incubated with varying doses of doxycycline for 4 days and then treated without and with 100 ng/ml CP-751,871. The experimental data was collected at different times as follows: T₀—before CP-751,871 treatment, analyzing cell number and β-arr1 expression, T₁₀ₘ—10 min after CP-751,871 addition for ERK activation, T₁₂ₕ—12 h after CP-751,871 treatment for IGF-1R expression and T₄₈ₕ, for cell viability. The data collected at T₀ validated the system and confirmed a doxycycline dose dependent decrease in β-arr1 (FIG. 6A). In addition, characterization of the system at T₀ demonstrated that the reduced β-arr1 induces an overall dose dependent loss of cell viability (FIG. 6A).

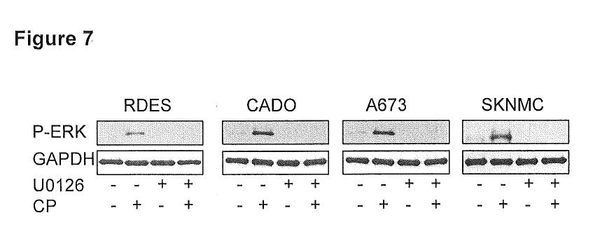

The proportion of cells inhibited by CP-751,871 treatment at T₄₈ₕ, described by the CP-751,871 ratio (CP-751,871 treated versus untreated) confirmed our hypothesis, showing that down to a certain level of β-arr1, the cells are more resistant to CP-751,871 (FIG. 6B). However, further decreases in β-arr1 reverse this protection, generating a bell shaped curve for cell viability through decreasing β-arr1 levels in all 5 cell lines. The data collected at T₁₂ₕ for IGF-1R expression (FIG. 6B) confirmed that β-arr1 downregulation prevents CP-751,871-induced IGF-1R degradation while results collected at T₁₀ₘ verified lower CP-751,871-induced ERK activation following β-arr1 inhibition (FIG. 6B). The relative changes between P-ERK and IGF-1R levels appear to correlate with the peak of the CP-751,871 cell viability inhibition rate (FIG. 6B), highlighting the dual role of β-arr1: receptor downregulation and signaling activation. This demonstrates that the biased activation of the ERK pathway by CP-751,871 protects, to a certain extent, against reduction of IGF-1R expression and cell viability by CP-751,871. To verify this mechanism, we selectively inhibited the ERK activation by adding MEK inhibitor U0126 ([1,4-d]amino-2,3-dicyano-1,4-bis(2-amionphe-nylthio butadiene)] MEK 1/2 inhibitor from Calbiochem) in combination with CP-751,871 stimulation to uncouple the protective effect of ERK activation from the detrimental IGF-1R downregulation. The MEK inhibitor effectively prevented CP-751,871-induced ERK activation (FIG. 7). Combining treatment of CP-751,871 with the MEK inhibitor demonstrated increased sensitivity over CP-751,871 alone, confirming the protective role of CP-751,871 biased agonism and validating one approach to circumvent it (FIG. 6C).

### EXAMPLE 2

To verify that the biased activation of the ERK pathway by anti-IGF-1R antibodies can protect against reduction of cell viability by IGF-1R antibodies, tests were carried out in different cancer cell lines, in which the ERK activation was selectively inhibited by adding MEK U0126 in combination with anti-IGF-1R antibodies. The experiments were designed to disengage the protective effect of ERK activation from the detrimental IGF-1R downregulation.

The effects of anti-IGF-1R antibodies/MEK inhibitor combination treatment on viability of different types of tumor cells were investigated using PrestoBlue cell viability assay (Invitrogen, CA, USA) according to manufacturer's instructions. (Table 3).

Cells were incubated in serum free medium for 12 h followed by pretreatment for 60 min without or with 0.5, 1, 3 or 5 μM of the ERK inhibitor U0126, and then treated without or with 100 ng/ml anti-IGF-1R antibodies (Figitumumab or αIR-3 (Calbiochem, US). Cells were incubated in 96 well tissue culture plates for 48 hours and cell viability was measured by PrestoBlue cell viability assay. Fluorescence was measured with excitation at 560 nm and emission at 590 nm. Cell number was calculated from standard curves of fluorescence measurements from known numbers of cells. Triplicate cultures were performed on all experiments. The inhibition induced by anti-IGF-1R antibodies treated cells was calculated as percentage of untreated cells.

The percentage inhibition values of anti-IGF-1R antibodies/MEK inhibitors combination determined by the viability of 18 different cancer cell lines after 48 h incubation are summarized in Table 3. Increased concentration of MEK inhibitors, enhanced responsiveness to anti-IGF-1R therapy in all cell lines demonstrated that this effect is cancer-type independent. In addition, similar effects were observed for both Figitumumab or αIR-3 antibodies, suggesting that protective biased MAPK/ERK activation is a common feature for receptor-downregulating anti-IGF-1R antibodies.

Without being bound by any particular theory, these results strongly suggest that the enhanced effects of the MAPK/ERK pathway inhibitor/anti-IGF-1R antibodies combination on cell proliferation and cell viability of malignant cells may be the result of inactivation of the IGF-1R biased MAPK/ERK signaling. Differences in response between cell lines may be due to differences in IGF-1R dependence, or the cellular uptake of the inhibitors; however, in all tested cancer types the combination therapy greatly enhanced the response.

TABLE 3
Effects of anti-IGF-1R targeting therapy on cell viability following inhibition
of biased MAPK/ERK activation in various cancer cell lines.

| | | Percentage inhibition of CP | | | | | Percentage inhibition of αIR- | | | | |
|---|---|---|---|---|---|---|---|---|---|---|---|
| | | treated cells (100 ng/ml) | | | | | 3 treated cells (100 ng/ml) | | | | |
| | | MEK inhibitor (U0126 μM) | | | | | MEK inhibitor (U0126 μM) | | | | |
| Origin | Cell line | 0 | 0.5 | 1 | 3 | 5 | 0 | 0.5 | 1 | 3 | 5 |
| Melanoma | BE | 22 | 27 | 32 | 45 | 55 | 12 | 17 | 25 | 32 | 35 |
| | DFB | 15 | 17 | 24 | 45 | 55 | 15 | 24 | 32 | 45 | 57 |
| | SKMel5 | 22 | 30 | 35 | 39 | 42 | 18 | 22 | 34 | 32 | 45 |
| | SKMel28 | 5 | 15 | 17 | 25 | 29 | 9 | 8 | 17 | 19 | 22 |
| Breast | MCF7 | 15 | 18 | 25 | 18 | 33 | 10 | 17 | 23 | 32 | 45 |
| carcinoma | ZR75-1 | 5 | 9 | 12 | 12 | 15 | 4 | 12 | 15 | 19 | 24 |
| | MDA-MB-231 | 8 | 12 | 15 | 22 | 32 | 10 | 16 | 22 | 23 | 34 |
| Hepatic | HEPG2 | 7 | 11 | 22 | 27 | 37 | 11 | 15 | 22 | 25 | 45 |
| carcinoma | | | | | | | | | | | |
| Liver | OMM 2.3 | 12 | 35 | 45 | 65 | 97 | 5 | 45 | 78 | 84 | 86 |
| metastasis of | OMM 2.5 | 7 | 33 | 65 | 87 | 89 | 3 | 32 | 45 | 55 | 68 |
| uveal melanoma | | | | | | | | | | | |
| Colon | Colo 205 | 21 | 28 | 34 | 45 | 67 | 19 | 22 | 37 | 56 | 78 |
| carcinoma | | | | | | | | | | | |
| Prostate | PC-3 | 22 | 24 | 32 | 45 | 52 | 18 | 27 | 34 | 45 | 43 |
| carcinoma | DU-145 | 18 | 22 | 34 | 54 | 77 | 9 | 12 | 25 | 35 | 45 |
| Lung cancer | A549 | 40 | 56 | 77 | 88 | 98 | 32 | 54 | 65 | 68 | 79 |
| | H1703 | 10 | 15 | 25 | 28 | 32 | 7 | 17 | 22 | 24 | 36 |
| Pancreatic | MIAPaCa2 | 27 | 35 | 55 | 78 | 89 | 14 | 23 | 32 | 41 | 57 |
| cancer | | | | | | | | | | | |
| Uveal | OCM-1 | 22 | 28 | 35 | 46 | 55 | 7 | 22 | 34 | 38 | 42 |
| melanoma | | | | | | | | | | | |
| Ovarian | SK-OV-3 | 17 | 25 | 35 | 55 | 67 | 5 | 15 | 18 | 25 | 36 |
| carcinoma | | | | | | | | | | | |

That which is claimed is:

1. A composition comprising at least one insulin growth factor 1 receptor (IGF-1R) antagonist and at least one mitogen activated protein kinase/extracellular signal-regulated kinase (MAPK/ERK) inhibitor.

2. The composition of claim 1, wherein the IGF-1R antagonist is an antibody directed against IGF-1R, a monoclonal antibody (mAb) directed against IGF-1R, an estrogen receptor (ER) inhibitor, an aromatase inhibitor, an AMP-activated protein kinase (AMPK) inhibitor, a small molecule tyrosine kinase inhibitor (TKI) or a small molecule tyrosine kinase (TKI)), or any combination thereof.

3. The composition of claim 2, wherein the monoclonal antibody is a humanized monoclonal antibody.

4. The composition of claim 3, wherein the humanized monoclonal antibody is selected from the group consisting of IMC-A12 (cixutumumab), R1507, MK-0646, AMG 479, AVE1642, CP-751,871 (figitumumab), and any combination thereof.

5. The composition of claim 1, wherein the MAPK/ERK pathway inhibitor is a Ras (Rat sarcoma) kinase inhibitor, Raf (Rapidly Accelerated Fibrosarcoma) kinase inhibitor, an ERK (Extracellular Signal-Regulated Kinase) inhibitor and/or a MEK (mitogen activated protein kinase kinase) inhibitor), or any combination thereof.

6. The composition of claim 5, wherein the Ras inhibitor is Tipifarnib (Zarnestra™, R115777.

7. The composition of claim 5, wherein the Raf inhibitor is selected from the group consisting of RAF265, XL281, PLX-4032, BAY 43-9006 (Nexavar®, Sorafenib tosylate), AAL-881, LBT-613, SB-590885, PLX-4720, L-779,450, SB-699393, and any combination thereof.

8. The composition of claim 5, wherein the MEK inhibitor is selected from the group consisting of CI-1040 (PD-184352), PD0325901, XL518, Selumetinib (AZD6244, ARRY-142886), RDEA119 (BAY 869766), PD098059, U0126, SL-327, and any combination thereof.

9. The composition of claim 1, further comprising a pharmaceutically acceptable excipient.

10. A pharmaceutical composition comprising the composition of claim 1 and a pharmaceutically acceptable excipient.

11. A method of treating cancer in a subject in need thereof comprising administering to said subject a therapeutically effective amount of at least one insulin, growth factor 1 receptor (IGF-1R) antagonist and at least one mitogen activated protein kinase/extracellular signal-regulated kinase (MAPK/ERK) inhibitor.

12. The method of claim 11, wherein the IGF-1R antagonist is an antibody directed against IGF-1R, a monoclonal antibody (mAb) directed against IGF-1R, an ER inhibitor, an aromatase inhibitor, an AMP-activated protein kinase (AMPK) inhibitor, a small molecule tyrosine kinase inhibitor (TKI) or a small molecule tyrosine kinase (TKI), or any combination thereof, and the MAPK/ERK pathway inhibitor is a Ras (Rat sarcoma) kinase inhibitor, Raf (Rapidly. Accelerated Fibrosarcoma) kinase inhibitor, an ERK (Extracellular Signal-Regulated Kinase) inhibitor and/or a MEK (mitogen activated protein kinase kinase) inhibitor.

13. The method of claim 12, wherein the monoclonal antibody is a humanized monoclonal antibody.

14. The method of claim 13, wherein the humanized monoclonal antibody is selected from the group consisting of IMC-A12 (cixutumumab), R1507, MK-0646, AMG 479, AVE1642, CP-751,871 (figitumumab), and any combination thereof.

15. The method of claim 12, wherein the MAPK/ERK inhibitor is a Ras/Raf (Rat sarcoma/Rapidly Accelerated Fibrosarcoma) kinase inhibitor, an ERK (Extracellular Signal-Regulated Kinase) inhibitor and/or a MEK (mitogen activated protein kinase kinase) inhibitor, and any combination thereof.

16. The method of claim 15, wherein the Ras inhibitor is Tipifarnib (Zarnestra™, R115777).

17. The method of claim 15, wherein the Raf inhibitor is selected from the group consisting of RAF265, XL281, PLX-4032, BAY 43-9006 (Nexavar®, Sorafenib tosylate), AAL-881, LBT-613, SB-590885, PLX-4720, L-779,450, SB-699393, and any combination thereof.

18. The method of claim 15, wherein the MEK inhibitor is selected from the group consisting of CI-1040 (PD-184352), PD0325901, XL518, Selumetinib (AZD6244, ARRY-142886), RDEA119 (BAY 869766), PD098059, U0126, SL-327, and any combination thereof.

19. The method of claim 11 wherein the cancer comprises cells that produce IGF-1R.

20. The method of claim 19, where the cancer is selected from the group consisting of Ewing's sarcoma, childhood sarcoma, colon cancer, melanoma, breast cancer, hepatic cancer, liver metastasis of cancer, liver metastasis of uveal melanoma, colon cancer, prostate cancer, lung cancer, pancreatic cancer, uveal melanoma, ovarian cancer, gastric cancer and any combination thereof.

21. The method of claim 20, wherein the MAPK/ERK pathway inhibitor is administered at substantially the same time with the IGF-1R antagonist.

22. The method of claim 20, wherein the MAPK/ERK pathway inhibitor is administered prior to the IGF-1R antagonist.

23. The method of claim 20, wherein the MAPK/ERK pathway inhibitor is administered after the IGF-1R antagonist.

24. The method of claim 20, wherein the MAPK/ERK inhibitor and the IGF-1R antagonist are administered in a single unit dosage form.

25. The method of claim 20, wherein the MAPK/ERK inhibitor and the IGF-1R antagonist are administered in multiple, separate unit dosage forms.


SEQUENCE LISTING (4 sequences)

SEQ ID NO: 1 - DNA, 21 bp, Artificial
  siRNA sequence
aaagccuucugugcugagaac

SEQ ID NO: 2 - DNA, 21 bp, Artificial
  Control non-targeting RNA duplex sequence
aauucuccgaacgugucacgu

SEQ ID NO: 3 - DNA, 66 bp, Artificial
  shRNA oligonucleotide sequence
tcgaggtctggataaggagatctatttcaagagaatagatctccttatccagattttttacgcgta

SEQ ID NO: 4 - DNA, 66 bp, Artificial
  shRNA oligonucleotide sequence
agcttacgcgtaaaaaatctggataaggagatctattctcttgaaatagatctccttatccagacc